(19) United States
(12) Patent Application Publication
(10) Pub. No.: US 2022/0371994 A1
(43) Pub. Date: Nov. 24, 2022

(54) CRYSTALLINE POLYMORPHIC FORM OF PSILOCIN

(71) Applicant: CANNA-CHEMISTRIES LLC, Vincennes, IN (US)

(72) Inventors: Nate Schultheiss, West Lafayette, IN (US); Travis Lee Houston, Lafayette, IN (US); Stephan D. Parent, West Lafayette, IN (US)

(21) Appl. No.: 17/737,543

(22) Filed: May 5, 2022

Related U.S. Application Data
(60) Provisional application No. 63/185,999, filed on May 7, 2021.

(51) Int. Cl. C07D 209/16

(52) U.S. Cl. CPC C07D 209/16; C07B 2200/13

(57) ABSTRACT

A crystalline polymorphic form of psilocin is disclosed. The beneficial and therapeutic uses of the crystalline form and of compositions containing the crystalline form are also disclosed. The disclosure sets out methods of making and characterizing the crystalline form.

### CROSS-REFERENCE TO RELATED APPLICATIONS

This application claims priority to U.S. Provisional Patent Application No. 63/185,999, filed May 7, 2021, which is incorporated herein by reference in their entirety.

### FIELD OF THE INVENTION

The invention relates to a crystalline form of psilocin and the processes for preparation thereof. The invention also relates to beneficial and therapeutic uses of the crystalline form and of compositions containing the crystalline form.

### BACKGROUND OF THE INVENTION

Crystalline forms of therapeutic drugs have been used to alter the physicochemical properties of the drug. Each crystalline form of a drug can have different solid-state (physical and chemical) properties which may be relevant for drug delivery. Crystalline forms often have better chemical and physical properties than corresponding non-crystalline forms such as the amorphous form. The differences in physical properties exhibited by a novel solid form of a drug (such as a polymorph or cocrystal or of the original drug) affect pharmaceutical parameters such as melting point, storage stability, compressibility, and density (relevant for formulation and product manufacturing), and dissolution rates and solubility (relevant factors in achieving suitable bioavailability).

Polymorphism, the occurrence of different crystalline forms, is a property of some molecules and molecular complexes. A single molecule, like psilocin, may give rise to a variety of polymorphs having distinct crystal structures and physical properties like melting point, thermal behaviors (e.g., measured by thermogravimetric analysis (TGA), or differential scanning calorimetry (DSC)), powder and single-crystal X-ray diffraction. One or more of these techniques may be used to distinguish different polymorphic forms of a compound. Obtaining a suitable crystalline form of a drug is often a necessary stage for many orally available drugs. Suitable crystalline forms possess the desired properties of a particular drug.

Psilocin (also known as 4-hydroxy DMT, 4-OH-DMT or 4-hydroxy-N,N-dimethyltryptamine) has a CAS number 520-53-6 and is a tryptamine alkaloid and a psychedelic substance. It is found in most psychedelic mushrooms with its phosphorylated counterpart psilocybin. In fact, once ingested, psilocybin is rapidly metabolized to psilocin, which then acts on serotonin receptors in the prefrontal cortex. The mind-altering effects of psilocin typically last from one to three hours, although to individuals under the influence of psilocin, the effects may seem to last much longer, since the drug can distort the perception of time. Psilocin has a low toxicity and has no significant effect on dopamine receptors, and reports of lethal doses of the drug are rare. As a therapeutic drug psilocin may be suitable for the treatment of diseases or disorders, or symptoms of diseases or disorders, such as anxiety, depression, psychotic disorder, Schizophrenia, major depressive disorder (MDD), post-traumatic stress disorder (PTSD), obsessive-compulsive disorder, headaches and withdrawal from opioids, cocaine, heroin, amphetamines, and nicotine.

One crystal structure of psilocin has been reported in the literature by T. J. Petcher and H. P Weber in 1974 published in the Journal of Chemical Society, Perkin Transactions 2 with a CCDC Ref Code of PSILIN. In fact, the field of psilocin crystalline materials appears to be a relatively unexplored landscape. There remains a need, therefore, for other psilocin crystalline forms.

### SUMMARY OF THE INVENTION

The invention relates to a crystalline polymorphic form of psilocin, 1. The molecular structure of psilocin is shown below:

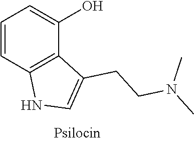

A crystalline polymorph of the invention may be characterized by: a single crystal X-ray diffraction structure having at least three peaks selected from the pattern 13.2, 13.9, 14.1, 16.6, 17.1, 20.0, 21.0, 21.1, 21.9, 22.4, and 25.4 ° 20±0.2° 20; a single crystal X-ray diffraction structure having at least three peaks selected from the pattern 13.2, 13.9, 14.1, 16.6, 17.1, 20.0, 21.1, 21.9, 22.4, 24.0, 25.4, 28.4, and 30.2 ° 20 ±0.2 ° 20; a calculated powder X-ray diffraction pattern substantially as shown in FIG. 2.

The invention further relates to a method of treating a disease, disorder or condition using psilocin the improvement comprising administering to a patient in need thereof a beneficial or therapeutically effective amount of psilocin, a composition, or a pharmaceutical composition of the invention.

### BRIEF DESCRIPTION OF THE FIGURES

FIG. 1 shows XRPD patterns of psilocin (as-received) and psilocin form 1.

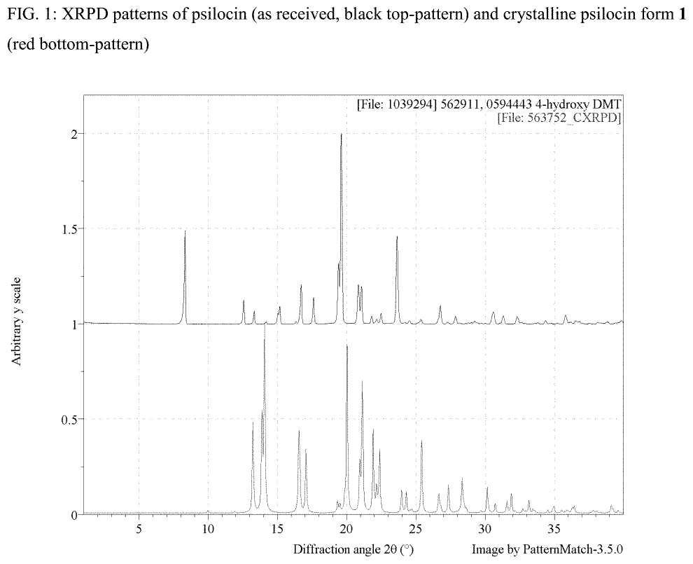

FIG. 2 shows a calculated XRPD pattern of psilocin form 1.

FIG. 3 shows an ORTEP drawing of psilocin form 1.

### DETAILED DESCRIPTION OF THE INVENTION

The invention relates to a new crystalline polymorphic form of psilocin. The psilocin form is not hydrated or solvated. The crystalline form was prepared and characterized by single crystal X-ray diffraction (SCXRD) as described in the examples below.

Therapeutic Uses of the Crystalline Psilocin Form

Psilocin (also known as 4-hydroxy DMT, 4-OH-DMT or 4-hydroxy-N,N-dimethyltryptamine) is known to be beneficial for human health. The invention also provides therapeutic and beneficial uses of the crystalline form and methods for its delivery, and compositions, such as pharmaceutical dosage forms, containing the crystalline form, to humans. The crystalline psilocin form of the invention may then be used to treat diseases, disorders, and conditions, such as those discussed above, and to provide beneficial treatment for humans. The invention therefore provides a method of treating a disease, disorder or condition using psilocin the improvement comprising administering to a patient in need thereof a beneficial or therapeutically effective amount of a crystalline psilocin form, a composition, or a pharmaceutical composition of the invention. Similarly, the invention relates to the use of psilocin to treat a disease, disorder or condition characterized by administering to a patient in need thereof a beneficial or therapeutically effective amount of a crystalline psilocin form, a composition, or a pharmaceutical composition of the invention.

The invention then also relates to the method of treating (or the use of crystalline psilosin form to treat) such a disease, disorder, or condition by administering to a human or animal patient in need thereof a therapeutically effective or beneficial amount of the crystalline psilocin form of the invention or of administering to a human or animal patient in need thereof a therapeutic composition containing the crystalline psilocin of the invention. The term “treatment” or “treating” means any treatment of a disease, disorder or condition in a mammal, including: preventing or protecting against the disease, disorder or condition, that is, causing the clinical symptoms not to develop; inhibiting the disease, disorder or condition, that is arresting or suppressing, the development of clinical symptoms; and/or relieving the disease, disorder or condition (including the relief of discomfort associated with the condition or disorder), that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” the disease, disorder or condition. The term “protection” is meant to include “prophylaxis.”

The crystalline psilocin of the invention may be administered at psilocin dosage levels of about 0.001 mg/kg to about 1.0 mg/kg, from about 0.01 mg/kg to about 0.5 mg/kg, or from about 0.1 mg/kg to about 0.20 mg/kg of subject body weight per day, one or more times a day, to obtain the desired effect. It will also be appreciated that, where appropriate, dosages smaller than 0.001 mg/kg or greater than 1.0 mg/kg (for example 1-2 mg/kg) can be administered to a subject in need thereof.

Compositions Containing the Crystalline Psilocin Form

The invention also relates to compositions, such as dietary supplement and pharmaceutical compositions, comprising a beneficial or therapeutically effective amount of the crystalline psilocin form according to the invention and a carrier, such as a pharmaceutically acceptable carrier (also known as a pharmaceutically acceptable excipient). The compositions and pharmaceutical dosage forms may be administered using any amount, any form of composition, dietary supplement or pharmaceutical composition and any route of administration a beneficial or therapeutically effective for treatment. As mentioned above, these pharmaceutical compositions are therapeutically useful to treat or prevent disorders such as those discussed above.

A pharmaceutical composition of the invention may be in any pharmaceutical dosage form known in the art which contains the crystalline psilocin form according to the invention. A composition, particularly a pharmaceutical composition, may be, for example, a tablet, a capsule, a liquid suspension, an injectable composition, a topical composition, an inhalable composition or a transdermal composition. The compositions, particularly pharmaceutical compositions generally contain, for example, about 0.1% to about 99.9% by weight of the crystalline psilocin form, for example, about 0.5% to about 99% by weight of the crystalline psilocin form of the invention and, for example, 99.5% to 0.5% by weight of at least one suitable carrier such as a pharmaceutically acceptable carrier and/or excipient. The composition may also be between about 5% and about 75% by weight of the crystalline psilocin form of the invention with the rest being at least one suitable pharmaceutical carrier and/or excipient, as discussed below.

The dosage form an appropriate pharmaceutically acceptable carrier or excipient in a desired dosage, as known by those of skill in the art, the pharmaceutical compositions of this disclosure can be administered to humans and other animals orally, rectally, parenterally, intravenously, intracisternally, intravaginally, intraperitoneally, topically (as by powders, ointments, or drops), buccally, as an oral or nasal spray, or the like, depending on the location and severity of the condition being treated. In one embodiment, the pharmaceutical composition is with an oral unit dosage form.

Compositions, particularly pharmaceutical compositions, of the invention include a beneficial or therapeutically effective amount of the crystalline psilocin form of the invention and a carrier such as a pharmaceutically acceptable carrier and/or excipient. Such pharmaceutically acceptable carriers and excipients, including, without limitation, binders, fillers, lubricants, emulsifiers, suspending agents, sweeteners, flavorings, preservatives, buffers, wetting agents, disintegrants, effervescent agents, and other conventional excipients and additives. The pharmaceutical compositions of the invention can thus include any one or a combination of the following: a pharmaceutically acceptable carrier or excipient; other medicinal agent(s); pharmaceutical agent(s); adjuvants; buffers; preservatives; diluents; and various other pharmaceutical additives and agents known in the art. These additional formulation additives and agents will often be biologically inactive and can be administered to humans without causing deleterious side effects or interactions.

Suitable additives may include, but are not limited to, microcrystalline cellulose, lactose, sucrose, fructose, glucose, dextrose, other sugars, di-basic calcium phosphate, calcium sulfate, cellulose, methylcellulose, cellulose derivatives, kaolin, mannitol, lactitol, maltitol, xylitol, sorbitol, other sugar alcohols, dry starch, dextrin, maltodextrin, other polysaccharides, or mixtures thereof.

In one embodiment of the invention the pharmaceutical composition is an oral unit dosage form containing a therapeutically effective amount of the crystalline psilocin form of the invention and a pharmaceutically acceptable carrier and/or excipient. Exemplary oral unit dosage forms for use in the present disclosure include tablets, capsules, powders, suspensions, and lozenges, which may be prepared by any conventional method of preparing pharmaceutical oral dosage forms. Oral unit dosage forms, such as tablets, may contain one or more pharmaceutically acceptable carriers and/or excipients such as known in the art as discussed above, including but not limited to, release modifying agents, glidants, compression aides, disintegrants, effervescent agents, lubricants, binders, diluents, flavors, flavor enhancers, sweeteners, and preservatives.

Tablet dosage forms may be partially or fully coated, sub-coated, uncoated, and may include channeling agents. The ingredients are selected from a wide variety of excipients known in the pharmaceutical formulation art. Depending on the desired properties of the oral dosage form, any number of ingredients may be selected alone or in combination for their known use in preparing such dosage forms as tablets.

### EXAMPLES

The following reagents and analytical methods were used to prepare and characterize the crystalline psilocin polymorphic form 1 of the invention.

Reagents: Psilocin was acquired from Cayman Chemical and used as received. All other chemicals were purchased from various suppliers and used without further purification.

X-ray Powder Diffraction (XRPD): Patterns were collected with a PANalytical X'Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5. The data acquisition parameters for each pattern are displayed above the image in the Data section of this report including the divergence slit (DS) before the mirror.

Single Crystal X-Ray Diffraction (SCXRD): Data were collected on a Rigaku SuperNova diffractometer, equipped with a copper anode microfocus sealed X-ray tube (Cu Kαλ=1.54184 Å) and a Dectris Pilatus3 R 200K hybrid pixel array detector. The structure was solved by direct methods using SHELXT (see Sheldrick, G. M. Acta Cryst. 015, A71, 3-8, herein incorporated by reference in its entirety). The remaining atoms were located in succeeding difference Fourier syntheses. The structure was refined using SHELXL-2014 (see Sheldrick, G. M. Acta Cryst. 015, A71, 3-8; and Sheldrick, G. M. Acta Cryst., 2008, A64, 112-122, both of which are herein incorporated by reference in its entirety). Hydrogen atoms residing on nitrogen and oxygen were refined independently. Hydrogen atoms residing on carbon were included in the refinement but restrained to ride on the atom to which they are bonded.

### Example 1

### Preparation of Crystalline Form of Psilocin 1

A turbid suspension was generated with 129.4 mg Psilocin (Cayman Chemical, lot 0594443) in 8 mL of chloroform (Macron Chemicals, lot 0000088119). The slight discoloration of the turbid suspension was improved by briefly sonicating while in contact with activated charcoal and then filtered through a 0.2-μm nylon filter. The solution was chilled over dry ice and a molar equivalent of 37% HCl was added (52 μL, Sigma-Aldrich, lot MKCM7837). An oil formed in the solution within approximately 3 minutes. The mixture was stored in the freezer for one day. The mixture was removed from the freezer and the solution was decanted from the oil. The decant was partially evaporated under a purge of nitrogen at room temperature until lamellar crystals formed.

### 1.1: XRPD Characterization of a Crystalline Form of Psilocin 1

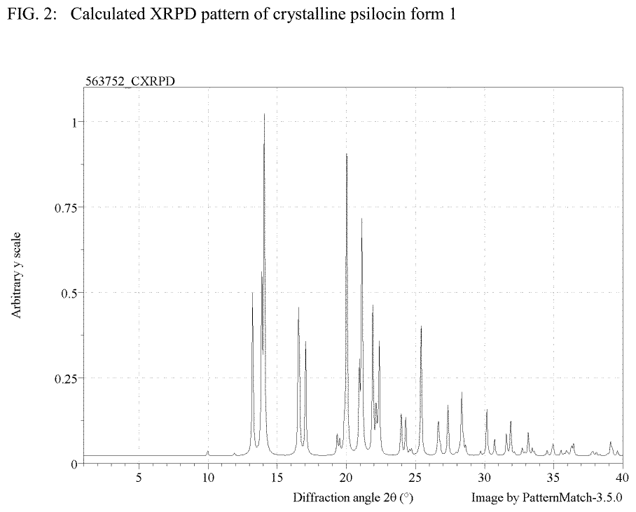

The calculated XRPD pattern of the crystalline form of psilocin 1 is shown in FIG. 2. Table 1 lists the angles, ° 2θ±0.2° 20, and d-spacing of the peaks identified in the calculated XRPD pattern of FIG. 2. For d-space listings, the wavelength used to calculate d-spacings was 1.5405929Å, the Cu-K_{α1} wavelength. The entire list of peaks, or a subset thereof, may be sufficient to characterize the form, as well as by an XRPD pattern substantially similar (that is, identifiable by one of ordinary skill using this method within experimental variations) to FIG. 2. For example, a crystalline form of psilocin of the invention may be characterized by a powder X-ray diffraction pattern having at least three peaks selected from 13.2, 13.9, 14.1, 16.6, 17.1, 20.0, 21.0, 21.1, 21.9, 22.4, and 25.4° 2θ±0.2° 20; or a powder X-ray diffraction pattern having at least three peaks selected from 13.2, 13.9, 14.1, 16.6, 17.1, 20.0, 21.1, 21.9, 22.4, 24.0, 25.4, 28.4, and 30.2° 2θ±0.2° 2θ.

TABLE 1

| 2-theta (deg) | d-spacing (Å) | Relative Intensity (I) |
|---|---|---|
| 10.0 | 8.86 | 1 |
| 11.9 | 7.43 | 1 |
| 13.2 | 6.69 | 47 |
| 13.9 | 6.37 | 54 |
| 14.1 | 6.29 | 100 |
| 16.6 | 5.35 | 43 |
| 17.1 | 5.19 | 34 |
| 19.3 | 4.59 | 6 |
| 19.5 | 4.55 | 5 |
| 20.0 | 4.43 | 89 |
| 21.0 | 4.24 | 29 |
| 21.1 | 4.20 | 69 |
| 21.9 | 4.05 | 44 |
| 22.2 | 4.01 | 16 |
| 22.4 | 3.97 | 34 |
| 24.0 | 3.71 | 12 |
| 24.3 | 3.66 | 11 |
| 24.6 | 3.62 | 2 |
| 24.7 | 3.60 | 2 |
| 25.4 | 3.50 | 38 |
| 26.7 | 3.34 | 10 |
| 26.8 | 3.33 | 4 |
| 27.4 | 3.26 | 15 |
| 28.0 | 3.19 | 1 |
| 28.4 | 3.15 | 19 |
| 28.6 | 3.12 | 3 |
| 29.7 | 3.01 | 1 |
| 30.2 | 2.96 | 14 |
| 30.7 | 2.91 | 5 |
| 31.6 | 2.83 | 6 |
| 31.9 | 2.84 | 10 |
| 32.2 | 2.78 | 1 |
| 32.7 | 2.73 | 2 |
| 32.9 | 2.72 | 1 |
| 33.2 | 2.70 | 7 |
| 33.5 | 2.68 | 2 |
| 33.6 | 2.67 | 1 |
| 34.5 | 2.60 | 2 |
| 35.0 | 2.56 | 4 |
| 35.5 | 2.52 | 2 |
| 35.8 | 2.51 | 1 |
| 35.9 | 2.50 | 2 |
| 36.3 | 2.47 | 3 |
| 36.4 | 2.46 | 4 |

### 1.2: SCXRD Characterization of a Crystalline Form of Psilocin 1

A colorless plate having approximate dimensions of 0.37×0.16×0.04 mm³, was mounted on a polymer loop in random orientation. The single crystal data and structure refinement parameters for the structure measured at 293 K are reported in Table 2, below. Cell constants and an orientation matrix for data collection were obtained from least-squares refinement using the setting angles of 2141 reflections in the range 4.9300° <θ<77.0740° . The space group was determined by the program CrysAlis Pro (see CrysAlisPro 1.171.38.41r (Rigaku Oxford Diffraction, 2015) to be P2₁/n (international tables no. 14).

The data were collected to a maximum diffraction angle (2θ) of 156.012° at room temperature.

Frames were integrated. A total of 4929 reflections were collected, of which 2329 were unique. Lorentz and polarization corrections were applied to the data. The linear absorption coefficient is 0.610 mm⁻¹ for Cu Kα radiation. An empirical absorption correction using CrysAlis Pro was applied. Transmission coefficients ranged from 0.675 to 1.000. A secondary extinction correction was applied. The final coefficient, refined in least-squares, was 0.0011(14) (in absolute units). Intensities of equivalent reflections were averaged. The agreement factor for the averaging was 1.94% based on intensity.

The structure was solved by direct methods using Shelxt (see Sheldrick, G. M. Acta Cryst. 015, A71, 3-8, herein incorporated by reference in its entirety). The remaining atoms were located in succeeding difference Fourier syntheses. The structure was refined using Shelxl-2014 (see Sheldrick, G. M. Acta Cryst. 015, A71, 3-8; and Sheldrick, G. M. Acta Cryst., 2008, A64, 112-122, both of which are herein incorporated by reference in their entirety). Hydrogen atoms residing on nitrogen and oxygen were refined independently. Hydrogen atoms residing on carbon were included in the refinement but restrained to ride on the atom to which they are bonded. The structure was refined in full-matrix least-squares by minimizing the function:

Σw(|F₀|²−|F_{c}|²)²

where the weight, w, is defined as 1/[σ²(F₀²)+(0.1280P)²+(0.9515P)], where P=(F₀²+2F_{c}²)/3.

Scattering factors were taken from the “International Tables for Crystallography” (see International Tables for Crystallography, Vol. C, Kluwer Academic Publishers: Dordrecht, The Netherlands, 1992, Tables 4.2.6.8 and 6.1.1.4., herein incorporated by reference in its entirety). Of the 2329 reflections used in the refinements, only the reflections with intensities larger than twice their uncertainty [I>2Σ(I)], 1754, were used in calculating the fit residual, R. The final cycle of refinement included 157 variable parameters, 2 restraints, and converged with respective unweighted and weighted agreement factors of:

R=Σ|F₀−F_{c}|/ΣF₀=0.0962

R_{w}={square root over ((Σw(F₀²−F_{c}²)²/Σw(F₀²)²))}=0.835

The standard deviation of an observation of unit weight (goodness of fit) was 1.10. The highest peak in the final difference Fourier had an electron density of 0.461 e/Å³. The minimum negative peak had a value of −0.471 e/Å³.

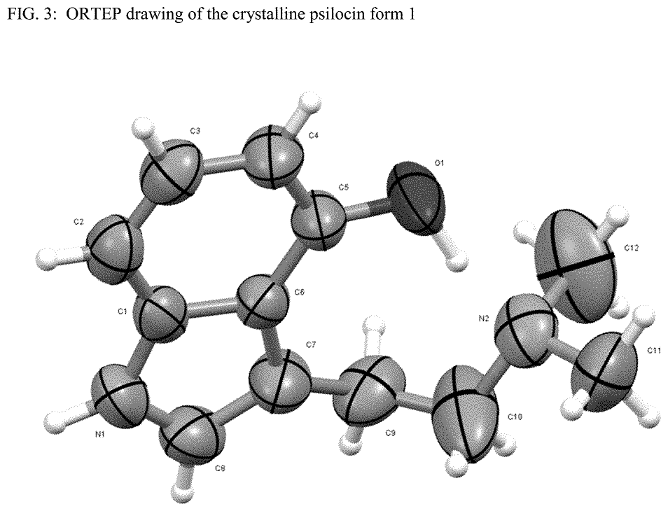

An ORTEP diagram of the crystalline psilocin form 1 at 293 K showing the numbering system employed is shown in FIG. 3. Anisotropic atomic displacement ellipsoids for the non-hydrogen atoms are shown at the 50% probability level and hydrogen atoms are displayed as spheres of arbitrary radius. The calculated MOD pattern based on the single crystal data and structure for the crystalline psilocin form 1 at 293 K is shown in FIG. 2.

TABLE 2

| | |
|---|---|
| Empirical formula | C₁₂H₁₆N₂O |
| Formula weight (g mol⁻¹) | 204.27 |
| Temperature (K) | 293(2) |
| Wavelength (Å) | 1.54184 |
| Crystal system | monoclinic |
| Space group | P2₁/n |
| Unit cell parameters | |
| a = 9.5331(7) Å | α = 90° |
| b = 8.9358(3) Å | β = 107.490(7)° |
| c = 14.0279(7) Å | γ = 90° |
| Unit cell volume (Å³) | 1139.73(11) |
| Cell formula units, Z | 4 |
| Goodness-of-fit on F² | S = 1.10 |
| Final residuals [I > 2σ(1)] | R = 0.0962, R_{w} = 0.2835 |
| Final residuals [all reflections] | R = 0.1134, R_{w} = 0.2982 |

What is claimed is:

1. A crystalline form of psilocin (4-hydroxy-N,N-dimethyltryptamine) exhibiting an X-ray powder diffraction pattern having at least three peaks at the following 2θ diffraction angles: 13.2, 13.9, 14.1, 16.6, 17.1, 20.0, 21.1, 21.9, 22.4, 24.0, 25.4, 28.4, and 30.2° 2θ±0.2° 2θ.

2. The crystalline form of claim 1, wherein the X-ray powder diffraction pattern has at least four peaks at the following 20 diffraction angles: 13.2, 13.9, 14.1, 16.6, 17.1, 20.0, 21.1, 21.9, 22.4, 24.0, 25.4, 28.4, and 30.2° 2θ±0.2° 2θ.

3. The crystalline form of claim 1, wherein the X-ray powder diffraction pattern has at least five peaks at the following 20 diffraction angles: 13.2, 13.9, 14.1, 16.6, 17.1, 20.0, 21.1, 21.9, 22.4, 24.0, 25.4, 28.4, and 30.2° 2θ±0.2° 2θ.

4. The crystalline form of claim 1, wherein the X-ray powder diffraction pattern has six peaks at the following 20 diffraction angles: 13.2, 13.9, 14.1, 16.6, 17.1, 20.0, 21.1, 21.9, 22.4, 24.0, 25.4, 28.4, and 30.2° 2θ±0.2° 2θ.

5. The crystalline form of claim 1, wherein the X-ray powder diffraction pattern has a peak at about 13.9° 2θ.

6. The crystalline form of claim 1, wherein the X-ray powder diffraction pattern has a peak at about 17.1° 2θ.

7. The crystalline form of claim 1, wherein the X-ray powder diffraction pattern has a peak at about 20.0° 2θ.

8. The crystalline form of claim 1, wherein the X-ray powder diffraction pattern has a peak at about 24.0° 2θ.

9. The crystalline form of claim 1, wherein the X-ray powder diffraction pattern has a peak at about 28.4° 2θ.

10. The crystalline form of claim 1, wherein the crystalline form has an X-ray powder diffraction pattern substantially as shown in FIG. 2.

11. The crystalline form of claim 1, wherein the crystalline form comprises a crystal with unit cell dimensions of α=9.5331(7) Å, b=8.9358(3) Å, c=14.0279(7) Å, α=90°, β=107.490(7)°, and γ=90°.

12. The crystalline form of claim 1, wherein the crystalline form comprises a crystal having unit cell parameters substantially equal to the following at 293K:

| Crystal system | monoclinic |
|---|---|
| Space group | P2₁/n |
| Unit cell parameters | |
| a = 9.5331(7) Å | α = 90° |
| b = 8.9358(3) Å | β = 107.490(7)° |
| c = 14.0279(7) Å | γ = 90° |
| Wavelength (Å) | 1.54184 |
| Unit cell volume (Å³) | 1139.73(11) |
| Cell formula units, Z | 4 |

13. A pharmaceutical composition comprising the crystalline form of claim 1, and at least one pharmaceutically acceptable carrier.

14. The pharmaceutical composition of claim 13, wherein the pharmaceutical composition is an oral formulation.

15. The pharmaceutical composition of claim 14, wherein the pharmaceutical composition is in an oral solid dosage form.

16. A method for preparing a crystalline form of claim 1, comprising:
preparing a suspension/solution of psilocin in an organic solvent;
contacting the suspension/solution of psilocin in the organic solvent with water, optionally containing an acid, to form a mixture comprising water/oil layers;
decanting the mixture to separate out the layer containing the psilocin; and
forming the crystalline form of psilocin from the layer containing the psilocin.

17. A method of treating a disease or disorder in a subject for which psilocin or psilocybin is indicated, comprising:
administering to the subject in need thereof a therapeutically effective amount of the crystalline form of claim 1.

18. The method of claim 17, wherein the disease or disorder is a psychiatric or psychotic disorder, neurocognitive disease or disorder, autism spectrum disorder (ASD), chronic pain, inflammatory disease or disorder, stroke, epilepsy disorder, amyotrophic lateral sclerosis (ALS), or combinations thereof.

19. The method of claim 18, wherein the disease or disorder is a psychiatric or psychotic disorder selected from the group consisting of attention-deficit hyperactivity disorder (ADHD), anxiety disorder, sleep-wake disorder, impulse-control, disruptive behavior, conduct disorder, depressive disorder, major depressive disorder (MDD), post-traumatic stress disorder (PTSD), obsessive-compulsive and related disorder, bipolar disorder, schizophrenia, and combinations thereof.

20. The method of claim 18, wherein the disease or disorder is a neurocognitive disease or disorder selected from the group consisting of Alzheimer's disease; Lewy Body Dementia;
Traumatic Brain Injury; Prion Disease; HIV Infection; Parkinson's disease; Huntington's disease;
alcohol-, smoking-, or drug-related condition or state; and combinations thereof.

21. A crystalline form of psilocin (4-hydroxy-N,N-dimethyltryptamine) exhibiting an X-ray powder diffraction pattern having at least three peaks at the following 20 diffraction angles: 13.2, 13.9, 14.1, 16.6, 17.1, 20.0, 21.0, 21.1, 21.9, 22.4, and 25.4° 2θ±0.2° 2θ.
